(19) United States
(12) Patent Application Publication
(10) Pub. No.: US 2024/0307435 A1
(43) Pub. Date: Sep. 19, 2024

(54) NASAL SPRAY COMPOSITIONS TO SUPPRESS CORONAVIRUS AND OTHER PATHOGENS

(71) Applicant: Courtland Imel, Farmers Branch, TX (US)

(72) Inventor: Courtland Imel, Farmers Branch, TX (US)

(21) Appl. No.: 18/123,226

(22) Filed: Mar. 17, 2023

(51) Int. Cl. A61K 33/30; A61K 9/00; A61K 33/06; A61K 47/10; A61K 47/12; A61K 47/18; A61P 31/14

(52) U.S. Cl. CPC A61K 33/30; A61K 9/0043; A61K 33/06; A61K 47/12; A61K 47/186; A61P 31/14

(57) ABSTRACT

A formulation for an antiviral nasal composition effective in reducing viable pathogens and, more particularly, effective as preventive and adjuvant therapy for nasal infections or diseases caused by other pathogens.

### BACKGROUND

### Field

The present disclosure relates generally to a nasal spray composition, and more particularly to a nasal composition and method that may be used in the prevention or treatment of respiratory tract infections.

### Background

In view of the bacterial and viral infections in the nasal passageway, the search for potential protective and therapeutic antiviral strategies is of particular and urgent interest.

Therefore, a nasal spray composition effective in reducing viable pathogens and, more particularly, effective as preventive and adjuvant therapy for nasal infections or diseases caused by other pathogens (e.g., virus, bacteria), is desired.

### SUMMARY

The following is a non-exhaustive listing of some aspects of the present techniques. These and other aspects are described in the following disclosure.

Some aspects include an antiviral nasal composition effective in reducing viable bacteria, and, more particularly, providing a protective effect as preventive and adjuvant therapy for nasal infections or diseases caused by other pathogens.

Some aspects include an antiviral nasal composition effective in reducing viable virus, and, more particularly, providing a protective effect as preventive and adjuvant therapy for nasal infections or respiratory diseases caused by other viruses.

Some aspects include an antibacterial nasal composition effective in reducing viable bacteria, and, more particularly, providing a protective effect as preventive and adjuvant therapy for respiratory diseases caused by bacteria.

Some aspects include an antiviral nasal composition, containing ionic compounds (e.g. zinc and magnesium), effective in modulating antiviral and antibacterial immunity and regulate inflammatory response. Some aspects include an antiviral nasal composition containing magnesium, effective in modulating antiviral and antibacterial immunity and regulating an inflammatory response. Some aspects include an antiviral nasal composition containing calcium, effective in modulating antiviral and antibacterial immunity and regulating an inflammatory response. Some aspects include an antiviral nasal composition containing rubidium, effective in modulating antiviral and antibacterial immunity and regulating an inflammatory response.

Several inventive embodiments of the present disclosure are described below.

### BRIEF DESCRIPTION OF THE DRAWINGS

The above-mentioned aspects and other aspects of the present techniques will be better understood when the present application is read in view of the following FIGURE in which like numbers indicate similar or identical elements:

FIG. 1 is a flow diagram showing a method of use of a nasal spray, in accordance with some embodiments of the present disclosure. FIG. 1 depicts a method 100 of use, in accordance with some embodiments, and may generally include obtaining the ingredients 10 (e.g. chemicals and components, like those discussed elsewhere herein), in accordance with some embodiments of the present disclosure, as shown in block 10, mixing these ingredients to obtain an antiviral nasal composition (e.g. nasal spray or nasal drop) as shown in block 12, packaging the antiviral nasal composition, to facilitate shipment, prolong shelf-life, ease of storage and facilitate administration, as shown in block 14, and nasally administering antiviral nasal composition to an individual (e.g. with a nasal spray) as shown in block 16.

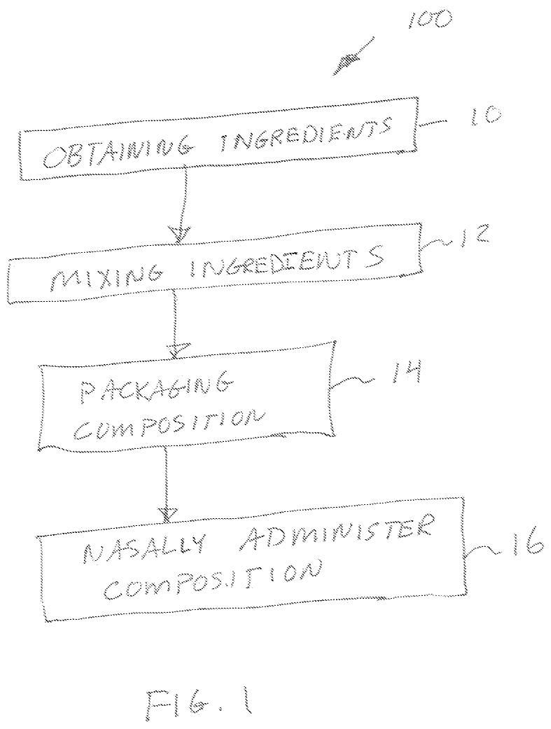

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION

To mitigate the problems described herein, the inventors had to both invent solutions and, in some cases just as importantly, recognize problems overlooked (or not yet foreseen) by others in prevention and treatment of bacterial and viral diseases in humans and animals. Further, because multiple problems are addressed, it should be understood that some embodiments are problem-specific, and not all embodiments address every problem with traditional systems described herein or provide every benefit described herein That said, improvements that solve various permutations of these problems are described below.

### Definitions

The term “effective amount,” as used herein, refers to the amount of active agent needed to achieve the desired therapeutic or prophylactic effect, such as an amount that is sufficient to reduce pathogen (e.g., bacteria, virus) burden, reduce symptoms (e.g., fever, coughing, sneezing, nasal discharge, and the like), reduce occurrence of infection, reduce viral replication, or improve or prevent deterioration of respiratory function, produce an effective serum concentration of a pharmaceutically active agent, increase mucociliary clearance, reduce total inflammatory cell count, or modulate the profile of inflammatory cell counts. The actual effective amount for a particular use can vary according to the particular nasal composition, the mode of administration, and the age, weight, general health of the subject, and severity of the symptoms or condition being treated. Suitable amounts of nasal composition to be administered, and dosage schedules for a particular subject can be determined by a clinician of ordinary skill based on these and other considerations.

The term “pharmaceutically acceptable excipient” as used herein means that the excipient can be taken into the nostrils of the nose with no significant adverse toxicological effects on the mucous membranes of the nasal passage or nasal cavity of the subject. Such excipients are generally regarded as safe (GRAS) by the U.S. Food and Drug Administration.

### Nasal Formulations

In some embodiments, a nasal composition is formulated (or adapted) for preventing or treating viral infection through nasal passages, such as in a form of nasal drops, nasal washes, nasal packing, or nasal sprays.

In some embodiments, the nasal composition may have a viscosity in the range of 2,500 to 40,000 centipoise. The viscosity of the composition is important because it facilitates maintenance of the composition in the nasal cavity in contact with the nasal membrane or with mucous on the membrane. When the viscosity is less than about 2,500 centipoise, the composition tends to be drawn by gravity out of the nasal cavity. If the viscosity is in excess of about 40,000 centipoise, the thickness of the composition interferes with the diffusion of ionic zinc through the composition to the nasal membrane.

In some embodiments, the nasal composition may include ionic multivalent metal components. An ionic multivalent metal component refers to any compound or composition that may release a multivalent metal cation. It may be a multivalent metal salt (including both organic salt and inorganic salt) or a composition comprising a non-salt multivalent metal compound and a solubilizing agent that causes the non-salt multivalent metal compound to release multivalent metal cations.

In some embodiments, an ionic multivalent metal component is soluble in water and releases free multivalent metal cations in quantities that are effective in treating or preventing at least one type of virus infection (in in-vitro tests), when dissolved in an aqueous solution. Multivalent metal cations include, Zn⁺⁺ Mg⁺⁺, Co⁺⁺, Ca⁺⁺, Cu⁺⁺, Fe⁺⁺, Fe⁺⁺⁺, Ni⁺⁺, Ni⁺⁺⁺, Al⁺⁺, Mn⁺⁺, Mn⁺⁺⁺, Ti⁺⁺, Ti⁺⁺⁺, Mo⁺⁺, and Mo⁺⁺⁺.

In some embodiments, the ionic multivalent metal component is present in an amount from about 0.01% to about 20% of the total weight of the nasal composition, such as from about 0.01% to about 10%, from about 0.02% to about 5%, or from about 0.05% to about 0.5% of the total weight of the nasal composition. In some embodiments, the ionic multivalent metal component is present in an amount from about 0.00001% to about 5%, such as from about 0.00001% to about 0.0001%, from about 0.0001% to about 0.001%, from about 0.001% to about 0.01%, from about 0.1% to about 5%, 0.00001% to about 5%, from about 0.0001% to about 5%, or from about 0.001% to about 5%.

In some embodiments, the ionic multivalent component is an ionic zinc component and is present in an amount (in chloride salt equivalent) at most about 0.1%, 0.2%, 0.3%, 0.4%, 0.5%, 0.6%, 0.7%, 0.8%, 0.9%, 1%, 1.2%, 1.4%, 1.6%, 1.8%, 2%, 2.2%, 2.4%, 2.6%, 2.8%, 3.0%, 3.5%, 4.0%, 4.5%, or 5% by weight of the nasal composition In some embodiments, the ionic zinc component is present in a minimal amount (e.g., homeopathic pharmacopeia up to a therapeutic concentration) (in chloride salt equivalent) for example less than about 5%, about 1%, about 0.01%, about 0.001%, about 0.0001%, about 0.00001%, or about 0.000001% by weight of the nasal composition. In some embodiments, the ionic zinc component is between about 0.00001% and about 5% by weight of the nasal composition.

In some embodiments, ionic zinc may refer to any compound or composition that may release zinc ion. It may be a zinc salt (including both organic salt and inorganic salt) or a composition comprising a non-salt zinc compound and a solubilizing agent that causes the non-salt zinc compound to release zinc ions.

In some embodiments, the nasal composition may include zinc, such as ionic zinc of divalent zinc ion, zinc salt of divalent zinc salt, zinc hydrate of zinc salt hydrate. Zinc salt may include inorganic zinc salt and organic zinc salt. Organic zinc salt may include zinc polycarboxylate, hydroxy carboxylic acid zinc and amino carboxylic acid zinc.

In some embodiments, the nasal composition may include ionic zinc. The ionic zinc may be provided by one or more zinc salts such as zinc citrate, zinc sulfate, zinc silicate, zinc lactate, zinc oxide and combinations thereof. The ionic zinc may be provided by one or more zinc salts such as zinc chloride, zinc citrate, zinc sulfate, zinc silicate, zinc lactate, zinc oxide, zinc phosphate, zinc chloride, zinc acetate, zinc chlorate, zinc perchlorate, zinc nitrate, zinc molybdate, zinc chromate, zinc ricinoleate, zinc carbonate, and combinations thereof. Is some embodiments, the ionic zinc is provided by zinc phosphate and/or zinc citrate.

In some embodiments, the nasal composition may include ionic zinc to modulate antiviral and antibacterial immunity and regulate inflammatory responses.

In some embodiments, the concentration of ionic zinc in the nasal composition may range from about 0.01% (100 ppm) to about 0.5% (5000 ppm), about from 0.02% (200 ppm) to about 0.35% (3500 ppm), or about from 0.05% (500 ppm) to about 0.2% (2000 ppm) by weight of the nasal composition.

In some embodiments, the ionic multivalent component is an ionic magnesium component and is present in an amount (in chloride salt equivalent) at most about 0.1%, 0.2%, 0.3%, 0.4%, 0.5%, 0.6%, 0.7%, 0.8%, 0.9%, 1%, 1.2%, 1.4%, 1.6%, 1.8%, 2%, 2.2%, 2.4%, 2.6%, 2.8%, 3.0%, 3.5%, 4.0%, 4.5%, or 5% by weight of the nasal composition. In some embodiments, the ionic magnesium component is present in a minimal amount (e.g., homeopathic pharmacopeia up to a therapeutic concentration) (in chloride salt equivalent) for example less than about 5%, about 1%, about 0.01%, about 0.001%, about 0.0001%, about 0 00001%, or about 0.000001% by weight of the nasal composition. In some embodiments, the ionic magnesium component is between about 0.00001% and about 5% by weight of the nasal composition.

In some embodiments, ionic magnesium may refer to any compound or composition that may release magnesium ion. It may be a magnesium salt (including both organic salt and inorganic salt) or a composition comprising a non-salt magnesium compound and a solubilizing agent that causes the non-salt magnesium compound to release magnesium ions.

In some embodiments, the nasal composition may include magnesium, such as ionic magnesium of divalent magnesium ion, magnesium salt of divalent magnesium salt, magnesium hydrate of magnesium salt hydrate. Magnesium salt may include inorganic magnesium salt and organic magnesium salt. Organic magnesium salt may include magnesium polycarboxylate, hydroxy carboxylic acid magnesium and amino carboxylic acid magnesium.

In some embodiments, the nasal composition may include ionic magnesium. The ionic magnesium may be provided by one or more magnesium salts such as magnesium citrate, magnesium sulfate, magnesium silicate, magnesium lactate, magnesium oxide and combinations thereof. The ionic magnesium may be provided by one or more magnesium salts such as magnesium chloride, magnesium citrate, magnesium sulfate, magnesium silicate, magnesium lactate, magnesium oxide, magnesium phosphate, magnesium chloride, magnesium acetate, magnesium chlorate, magnesium perchlorate, magnesium nitrate, magnesium molybdate, magnesium chromate, magnesium ricinoleate, magnesium carbonate, and combinations thereof. In some embodiments, the ionic magnesium is provided by magnesium phosphate and/or magnesium citrate.

In some embodiments, the nasal composition may include ionic magnesium to modulate antiviral and antibacterial immunity and regulate inflammatory responses.

In some embodiments, the concentration of ionic magnesium in the nasal composition may range from about 0.01% (100 ppm) to about 0.5% (5000 ppm), about from 0.02% (200 ppm) to about 0.35% (3500 ppm), or about from 0.05% (500 ppm) to about 0.2% (2000 ppm) by weight of the nasal composition.

In some embodiments, the ionic multivalent component is an ionic rubidium component and is present in an amount (in chloride salt equivalent) at most about 0.1%, 0.2%, 0.3%, 0.4%, 0.5%, 0,6%, 0.7%, 0.8%, 0.9%, 1%, 1.2%, 1.4%, 1.6%, 1.8%, 2%, 2.2%, 2.4%, 2.6%, 2.8%, 3.0%, 3.5%, 4.0%, 4.5%, or 5% by weight of the nasal composition. In some embodiments, the ionic rubidium component is present in a minimal amount (e.g., homeopathic pharmacopeia up to a therapeutic concentration) (in chloride salt equivalent) for example less than about 5%, about 1%, about 0.01%, about 0.001%, about 0.0001%, about 0.00001%, or about 0.000001% by weight of the nasal composition. In some embodiments, the ionic rubidium component is between about 0.00001% and about 5% by weight of the nasal composition.

In some embodiments, ionic rubidium may refer to any compound or composition that may release rubidium ion. It may be a rubidium salt (including both organic salt and inorganic salt) or a composition comprising a non-salt rubidium compound and a solubilizing agent that causes the non-salt rubidium compound to release rubidium ions.

In some embodiments, the nasal composition may include rubidium, such as ionic rubidium of divalent rubidium ion, rubidium salt of divalent rubidium salt, rubidium hydrate of rubidium salt hydrate. Rubidium salt may include inorganic rubidium salt and organic rubidium salt. Organic rubidium salt may include rubidium polycarboxylate, hydroxy carboxylic acid rubidium and amino carboxylic acid rubidium.

In some embodiments, the nasal composition may include ionic rubidium. The ionic rubidium may be provided by one or more rubidium salts such as rubidium citrate, rubidium sulfate, rubidium silicate, rubidium lactate, rubidium oxide and combinations thereof. The ionic rubidium may be provided by one or more rubidium salts such as rubidium chloride, rubidium citrate, rubidium sulfate, rubidium silicate, rubidium lactate, rubidium oxide, rubidium phosphate, rubidium chloride, rubidium acetate, rubidium chlorate, rubidium perchlorate, rubidium nitrate, rubidium molybdate, rubidium chromate, rubidium ricinoleate, rubidium carbonate, and combinations thereof. Is some embodiments, the ionic rubidium is provided by rubidium phosphate and/or rubidium citrate.

In some embodiments, the nasal composition may include ionic rubidium to modulate antiviral and antibacterial immunity and regulate inflammatory responses.

In some embodiments, the concentration of ionic rubidium in the nasal composition may range from about 0.01% (100 ppm) to about 0.5% (5000 ppm), about from 0.02% (200 ppm) to about 0.35% (3500 ppm), or about from 0.05% (500 ppm) to about 0.2% (2000 ppm) by weight of the nasal composition.

In some embodiments, the ionic multivalent component is an ionic calcium component and is present in an amount (in chloride salt equivalent) at most about 0.1%, 0.2%, 0.3%, 0.4%, 0.5%, 0.6%, 0.7%, 0.8%, 0.9%, 1%, 1.2%, 1.4%, 1.6%, 1.8%, 2%, 2.2%, 2,4%, 2.6%, 2.8%, 3.0%, 3.5%, 4.0%, 4.5%, or 5% by weight of the nasal composition. In some embodiments, the ionic calcium component is present in a minimal amount (e.g., homeopathic pharmacopeia up to a therapeutic concentration) (in chloride salt equivalent) for example less than about 5%, about 1%, about 0.01%, about 0.001%, about 0.0001%, about 0.00001%, or about 0.000001% by weight of the nasal composition. In some embodiments, the ionic rubidium component is between about 0.00001% and about 5% by weight of the nasal composition.

In some embodiments, ionic calcium may refer to any compound or composition that may release calcium ion. It may be a calcium salt (including both organic salt and inorganic salt) or a composition comprising a non-salt calcium compound and a solubilizing agent that causes the non-salt calcium compound to release calcium ions.

In some embodiments, the nasal composition may include calcium, such as ionic calcium of divalent calcium ion, calcium salt of divalent calcium salt, calcium hydrate of calcium salt hydrate. Calcium salt may include inorganic calcium salt and organic calcium salt. Organic calcium salt may include calcium polycarboxylate, hydroxy carboxylic acid calcium and amino carboxylic acid calcium.

In some embodiments, the nasal composition may include ionic calcium. The ionic calcium may be provided by one or more calcium salts such as calcium citrate, calcium sulfate, calcium silicate, calcium lactate, calcium oxide and combinations thereof. The ionic calcium may be provided by one or more calcium salts such as calcium chloride, calcium citrate, calcium sulfate, calcium silicate, calcium lactate, calcium oxide, calcium phosphate, calcium chloride, calcium acetate, calcium chiorate, calcium perchlorate, calcium nitrate, calcium molybdate, calcium chromate, calcium ricinoleate, calcium carbonate, and combinations thereof. Is some embodiments, the ionic calcium is provided by calcium phosphate and/or calcium citrate.

In some embodiments, the nasal composition may include ionic calcium to modulate antiviral and antibacterial immunity and regolate inflammatory responses.

In some embodiments, the concentration of ionic calcium in the nasal composition may range from about 0.01% (100 ppm) to about 0.5% (5000 ppm), about from 0.02% (200 ppm) to about 0.35% (3500 ppm), or about from 0.05% (500 ppm) to about 0.2% (2000 ppm) by weight of the nasal composition.

In some embodiments, the nasal composition may include one or more pharmaceutically useful excipients, including pH adjusting agents, pH buffer, viscosity modifiers, osmotic agents, flavor, carbohydrate (e.g., to act as a sweetener), preservatives (e.g., metal chelators), adhesives and colorants. The selection and use of each agent are determined based on the practices known to those skilled in art.

In some embodiments, the nasal composition may further include various types of amino acids. Examples of amino acids include the common natural amino acids, such as lysine, arginine, histidine, glycine, serine, threonine, asparagine, glutamine, cysteine, selenocysteine, proline, alanine, valine, isoleucine, leucine, methionine, phenylalanine, tyrosine, tryptophan, aspartic acid, and glutamic acid. In some embodiments the amino acid is a neutral or acidic amino acid, such as glycine. In some embodiments, the nasal composition includes one or more standard or non-standard amino acids, such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, lanthionine, 2-aminoisobutyric acid, dehydroalanine, gamma-aminobutyric acid, cystine, desmosine, isodesmosine, hydroxyproline, hydroxylysine, gamma-carboxyglutamate, phosphoserine, phosphothreonine, and phosphotyrosine.

In some embodiments, the nasal composition may farther include a buffering agent, for example, a sodium phosphate buffer (e.g., sodium dihydrogen phosphate and disodium hydrogen phosphate).

In some embodiments, the nasal composition may further include one or more surfactants, for example selected from the group consisting of anionic, cationic, zwitterionic, and nonionic surfactants, and mixtures thereof.

In some embodiments, the nasal composition may further include an antioxidant. Antioxidants may be vitamins (e.g. vitamin A, C or E), polyphenols, tocopherols, ethylene-diaminetetraacetic acid, butylhydroxytoluene (BHT), and propyl gallate.

In some embodiments, the nasal composition may contain a moisturizing agent. Examples of moisturizing agents include propylene glycol, glycerin and the like. The amount of moisturizing agent may be from about 0 to 10%, 1.00 to 4.00%, or 1.5 to 3.50% by weight/volume of the total nasal composition.

In some embodiments, the nasal composition may further include a thickening agent, such as xanthan gum or carrageenan, vermiculite thickener, and combinations thereof.

In some embodiments, the nasal composition may contain aldehydes and/or ketones. The nasal composition may contain one or more aldehydes, such as, for example, formaldehyde, acetaldehyde, propionaldehyde, butyraldehyde, benzaldehyde, cinnamaldehyde, vanillin, tolualdehyde, furfural, retinaldehyde, or combinations thereof. The nasal composition may contain one or more ketones, for example, propanone, butanone, pentanone, hexanone, heptanone, octanone, nonanone, decanone, and combinations thereof.

In some embodiments, the nasal composition may further include an anti-inflammatory agent. Anti-inflammatory agents may be selected from the group consisting of betaglycyrrhetinic acid, enoxolone, salicylic acid, azulene, ginkgo biloba, witch hazel, corticosteroid, NSAID, and salts thereof.

In some embodiments, the nasal composition may further include one or more antibacterial agents, for example, an antibacterial agent selected from the group consisting of halogenated diphenyl ethers (e.g. triclosan, herbal extracts, and essential oils.) For example, rosemary extract, tea extract, magnolia extract, thymol, menthol, cincole, geranol, carvacrol, citral, camphorol, catechol, bayberry Methyl ester, epigallocatechin gallate, epigallocatechin, gallic acid, miswak extract, sea buckthorn extract.

In some embodiments, the nasal composition may include a humectant selected from the group consisting of glycerin, propylene glycol, hydrogenated glucose syrup, polyethylene glycol-14 (PEG-14), polyethylene glycol-18 (PEG-18), polyethylene glycol-55 (PEG-55), polyethylene glycol-100 (PEG-100), polyethylene glycol-135 (PEG-135), polyethylene glycol-180 (PEG-180), polyethylene glycol-200 (PEG-4), polyethylene glycol-240 (PEG-240), polyethylene glycol-300 (PEG-6), polyethylene glycol-400 (PEG-8), polyethylene glycol-450 (PEG-9), polyethylene glycol-500 (PEG-10), polyethylene glycol-600 (PEG-12), polyethylene glycol-1540 (PEG-32), by ethylene glycol-2000 (PEG-40 or PEG-2M), polyethylene glycol-3000 (PEG-60), polyethylene glycol-4000 (PEG-75), polyethylene glycol-6000 (PEG-150), polyethylene glycol-9000 (PEG-9M or PEG-200), polyethylene glycol-20000 (PEG-20M or PEG-350), polyethylene glycol-600000 (PEG-14M), propylene glycol (PG), glycerol (glycerin), erythritol, xylitol, sorbitol, mannitol, Jactitol, and hydrogenated starch hydrolysates.

In some embodiments, the pH of the nasal composition is from about pH 2 to about pH 8. In some embodiments, the pH of the nasal composition is from about pH 2 to about pH 10. In some embodiments, the pH of the nasal composition is from about pH 4 to about pH 8. In some embodiments, the pH of the nasal composition is from about pH 4 to about pH 10. The pH of the nasal composition may be about 2, about 3, about 4, about 5, about 6, about 7, about 8, about 9, or about 10. In some embodiments, the pH of the nasal composition is about pH 6. The preferred pH of the formulation can differ depending on the specific viral indication that is being treated. For example, the pH needed to effectively kill a particular virus may be more or less acidic than the pH needed to effectively kill a different virus.

In one embodiment, the nasal composition comprises water, sodium chloride, sodium phosphate dibasic, potassium chloride, rubidium chloride, calcium chloride dihydrate, and magnesium chloride hexahydrate.

In some embodiments the nasal composition comprises water, sodium chloride, sodium phosphate dibasic, potassium chloride, rubidium chloride, calcium chloride dihydrate, magnesium chloride hexahydrate, benalkonium chloride, and citric acid.

In some embodiments, the nasal composition may be selected from the group consisting of water, Sodium Chloride, Sodium Phosphate Dibasic, Potassium Chloride, Rubidium Chloride, Calcium Chloride Dihydrate, magnesium Chloride Hexahydrate, Benalkonium Chloride (50%), and Citric Acid.

The amount of water in the nasal composition may be about 60% to about 90% of the nasal composition by weight, for example the amount of water in the nasal composition may be about 60%, 65%, 70%, 72%, 74%, 76%, 78%, 80%, 82%, 84%, 86%, 88%, or 90% of the nasal composition by weight.

The amount of potassium chloride in the nasal composition may be about 0.5% to about 2% of the nasal composition by weight, for example, the amount of potassium chloride in the nasal composition may be about 0.5%, 0.6%, 0.7%, 0.8%, 0.9%, 1.0%, 1.2%, 1.4%, 1.6%, 1.8%, or 2% of the nasal composition by weight.

The amount of rubidium chloride in the nasal composition may be about 0.004% to about 0.016% of the nasal composition by weight, for example, the amount of rubidium chloride in the nasal composition may be about 0.004%, 0.005%, 0 006%, 0.008%, 0.009%, 0.010%, 0.012%, 0.014%, or 0.016% of the nasal composition by weight.

The amount of calcium chloride dihydrate in the nasal composition may be about 0.0001% to about 0.0005% of the nasal composition by weight, for example, the amount of calcium chloride dihydrate in the nasal composition may be about 0.0001%, 0.00015%, 0.00020%, 0.00022%, 0.00023%, 0.00025%, 0.00030%, 0.00040%, or 0.00050% of the nasal composition by weight.

The amount of zinc chloride in the nasal composition may be about 0.00010% to about 0.00020% of the nasal composition by weight, for example, the amount of zinc chloride in the nasal composition may be about 0.00010%, 0.00011%, 0.00012%, 0.00013%, 0.00014%, 0.00015%, 0.00016%, 0.00017%, 0.00018%, 0.00019%, or 0.00020% of the nasal composition by weight.

In some embodiments, the nasal composition may further include an antiviral agent. Antiviral agents may be selected from the group consisting of abacavir, acyclovir, acyclovir, adefovir dipivoxil, amantadine, amprenavir, ampligen, arbidol, atazanavir, atripura, voseprevir, Cidofovir, combivir, darunavir, delavirdine, didanosine, docosanol, edoxine, efavirenz, emtricitabine, enfuvirtide, entecavir, entry inhibitor, famciclovir, fixed dose combination (antiretrovirus), fomivirsen, phosamprenavir, foscal Net, phosphonet, fusion inhibitor, ganciclovir, ivacitabine, immunovir, idoxuridine, imiquimod, indinavir, inosine, in Glase inhibitor, interferon type III, interferon type II, interferon type I, interferon, lamivudine, lopinavir, lobilide, maraviroc, morodidin, methisazone, nelfinavir, nevirapine, nexavir, nucleoside analog (nucleoside analog reverse transcriptase inhibitor), oseltamivir, Peginterferon alpha-2a, peginterferon alpha, pencyclovir, peramivir, pleconaril, podophyllox, podophyllotoxin, protease inhibitor, pyramidine, raltegravir, reverse transcriptase inhibitor, ribavirin, rimantadine, ritonavir, saquinavir, stavudine, synergistic enhancer (Anti-retrovirus), tea tree oil, tenofovir, tenofovir disoprox Includes syl, tipranavir, trifluridine, tridivir, tromantadine, turvada, valacyclovir, valganciclovir, bicrivirok, vidarabine, viramidine, zalcitabine, zanamivir, and zidovudine.

### Methods of Treatment

The disclosure also relates to methods of treating and preventing respiratory diseases (e.g., respiratory disease caused by viral and/or bacterial infection). The nasal compositions described herein can be administered to a subject in need thereof for the treatment of respiratory diseases, such as respiratory caused by viral infections (e.g., coronavirus, influenza virus, parainfluenza virus, respiratory syncytial virus, rhinovirus, adenovirus, metapneumovirus, coxsackie virus, echo virus, herpes virus, cytomegalovirus, and the like), or bacterial infections (e.g., Streptococcus pneumoniae, which is commonly referred to as pneumococcus, Staphylococcus aureus, Burkholderis ssp., Streptococcus agalactiae, Haemophilus influenzae, Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Mycobacterium tuberculosis, Bordetella pertussis, and the like).

In a particular aspect, the nasal compositions described herein are administered to a subject in thereof for treatment or prevention of the common cold (e.g., a coronavirus), a COVID19 infection (e.g. caused by SARS-Cov-2 or a variant thereof), or the flu (e.g., influenza virus).

In some embodiments, nasally administering or nasal administration includes administering the compositions into nostrils of the nose to the mucous membranes of the nasal passage or nasal cavity of the subject. Such formulations may be administered, for example, as a nasal spray, nasal inhaler, nasal drop, aerosol, propellants, pressured dispersion, aqueous aerosol, nebulizer, nasal suspension, instillation, nasal gel, nasal ointment and nasal cream by aid of any new or old type device. Administration of compositions of the present disclosure may also take place using a nasal tampon or nasal sponge containing the compositions.

In some embodiments, the nasal composition may be delivered using a nasal device that supplies a fixed (measured) volume or an amount (dose) of a nasal composition after each actuation. Examples of measured dose devices for nasal administration include an atomizer, sprayer, dropper, squeeze tube, squeeze spray bottle, pipette, blister, nasal cannula, measured dose device, spray inhaler nasal, bi-directional breathing-powered delivery device, pump sprayer, pre-compression measured dose spray pump, monopulverization pump, bipulverization pump and pressurized measured dose device. The administration device may be a disposable single-dose device, reusable single-dose device, disposable multi-dose device or reusable multi-dose device.

Suitable dosing to provide the desired therapeutic effect can be determined by a clinician based on the severity of the condition (e.g., infection), overall well-being of the subject and the subject's tolerance to nasal compositions and other considerations. Based on these and other considerations, a clinician can determine appropriate doses and intervals between doses. Generally, nasal compositions are administered once, twice or three times a day, as needed

Formulations may be conveniently presented in unit dosage form and may be prepared by any methods known in the art.

In some embodiments, the nasal composition may decrease the concentration of a viral stock of SARS-COV-2 by more than 90%, 95%, or 99% after an hour incubation.

In some embodiments, the nasal composition may decrease the concentration of a viral stock of SARS-COV-2 by more than 90%, 95%, or 99% after less than 1, 5, 10, 30, or 60 minutes incubation.

Exemplification is presented below that describes a preliminary in vitro study on anti-SARS-COV-2 viricidal activity of the nasal compositions with the present techniques.

### EXEMPLIFICATION

### Example 1: In Vitro Study

The anti-SARS-COV-2 viricidal activity of mouthwash and nasal wash/spray PDR formulations was tested in an in vitro study where each formulation was mixed with a viral stock of SARS-CoV-2, incubated for 1 hour, and the Median Tissue Culture Infectious Dose (TCID5) was compared to a control of phosphate buffered saline (PBS). In total, 2 samples of the mouthwash, 2 samples of the nasal wash, and 2 samples of the nasal spray were tested. When compared to the PBS control, there was an average of 3.12 log (99.92%) decrease in viral concentration following incubation with the mouthwash, a 2.62 log (99.76%) decrease in viral concentration when incubated with the nasal spray, and a 2.26 log (99.76%) decrease in viral concentration when incubated with the nasal wash.
Sample 1: Batch No. B20121-001 a citric acid/potassium citrate buffered solution with a final pH of 6.73
Sample 2: Batch No. B20121-002 a citric acid/sodium phosphate buffered solution with a final pH of 7.82

PDR Nasal spray and nasal wash included water, sodium chloride, sodium phosphate dibasic, potassium chloride, rubidium chloride, calcium chloride dihydrate, magnesium chloride hexahydrate, benalkonium chloride (50%), and citric acid

| | | | | | | Log | |
|---|---|---|---|---|---|---|---|
| | Time of | | | | Average | Reduction | Percent |
| Sample | Contact | | Log10 | Average | Log10 | to Virus | Log |
| Name | (minutes) | TCID50/mL | TCID50/ml | TCID50/ml | TCID50/ml | Controls | Reduction |
| MW1-1h | 60 | 1.47E+03 | 3.17 | 1.18E+03 | 3 | 3.12 | 99.92% |
| MW2-1h | 60 | 3.16E+02 | 2.5 | | | | |
| MW1-1h | 60 | 1.47E+03 | 3.17 | | | | |
| MW2-1h | 60 | 1.47E+03 | 3.17 | | | | |
| NS1-1h | 60 | 3.16E+03 | 3.5 | 3.16E+03 | 3.5 | 2.62 | 99.76% |
| NS2-1h | 60 | 3.16E+03 | 3.5 | | | | |
| NS1-1h | 60 | 3.16E+03 | 3.5 | | | | |
| NS2-1h | 60 | 3.16E+03 | 3.5 | | | | |
| NW1-1h | 60 | 3.16E+03 | 3.5 | 3.16E+03 | 3.5 | 2.62 | 99.76% |
| NW2-1h | 60 | 3.16E+03 | 3.5 | | | | |
| NW1-1h | 60 | 3.16E+03 | 3.5 | | | | |
| NW2-1h | 60 | 3.16E+03 | 3.5 | | | | |
| PBS1-1h | 60 | 2.08E+06 | 6.32 | 1.42E+06 | 6.12 | NA | NA |
| PBS2-1h | 60 | 1.47E+06 | 6.17 | | | | |
| PBS1-1h | 60 | 6.81E+05 | 5.83 | | | | |
| PBS2-1h | 60 | 1.47E+06 | 6.17 | | | | |

It should be understood that the description and the FIGURES are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating.” “determining” or the like refer to actions or processes of a specific apparatus, such as a special purpose computer or a similar special purpose electronic processing/computing device. The terms “first”, “second”, “third.” “given” and so on, if used in the claims, are used to distinguish or otherwise identify, and not to show a sequential or numerical limitation.

What is claimed is:

1. A nasal composition comprising one or more ionic compounds in an amount effective to modulate antiviral and/or antibacterial immunity and regulate inflammatory response.

2. The nasal composition of claim 1, wherein the one or more ionic compounds is zinc, magnesium, or a combination thereof.

3. The nasal composition of claim 2, wherein the one or more ionic compounds is zinc.

4. The nasal composition of claim 2, wherein the one or more ionic compounds is magnesium.

5. The nasal composition of claim 1, further comprising a moisturizing agent.

6. The nasal composition of claim 5, wherein the moisturizing agent is propylene glycol, glycerin, or a combination thereof.

7. The nasal composition of claim 1, wherein the nasal composition has a viscosity between 100 to 1000 centipoise.

8. The nasal composition of claim 1, wherein the one or more ionic compounds is an ionic multivalent metal component.

9. The nasal composition of claim 8, wherein the ionic multivalent metal component is configured to release zinc or magnesium multivalent metal cations.

10. The nasal composition of claim 8, wherein the ionic multivalent metal component is present in an amount from about 0.0001% to about 5% of the total weight of the nasal composition.

11. The nasal composition of claim 1, comprising an amount of ionic zinc effective to modulate antiviral and/or antibacterial immunity and regulate inflammatory responses.

12. The nasal composition of claim 1, further comprising an excipient.

13. The nasal composition of claim 1, further comprising an amino acid.

14. The nasal composition of claim 1, further comprising a buffering agent.

15. The nasal composition of claim 1, further comprising a surfactant.

16. The nasal composition of claim 1, further comprising an antioxidant

17. The nasal composition of claim 1, further comprising an anti-inflammatory agent.

18. The nasal composition of claim 1, further comprising a thickening agent.

19. The nasal composition of claim 1, wherein the pH of the nasal composition is from about pH 2 to about pH 10.

20. The nasal composition of claim 1, wherein the nasal composition comprises water, sodium chloride, sodium phosphate dibasic, potassium chloride, rubidium chloride, calcium chloride dihydrate, magnesium chloride hexahydrate, benalkonium chloride, and citric acid.

21. The nasal composition of claim 1, wherein the nasal composition is formulated for delivery as nasal spray, nasal gel, nasal ointment, or nasal cream.

22. A method of treating a respiratory tract infection comprising administering to nasal passages of a subject in need thereof an effective amount of a nasal composition comprising one or more ionic compounds is zinc, magnesium, or a combination thereof.

23. The method of claim 22, wherein the respiratory tract infection is a viral infection.

24. The method of claim 23, wherein the viral infection is a SARS-COV-2 viral infection.

25. A method of preventing a nasal infection comprising, administering an effective amount of a nasal composition comprising one or more ionic compounds including zinc, magnesium, or a combination thereof, to nasal passages of a subject in need thereof.

26. A method of decreasing viral concentration in a viral stock of SARS-COV-2 comprising, incubating the viral stock with a nasal composition comprising one or more ionic compounds including zinc, magnesium, or a combination thereof, for a time period of 60 minutes or less.

27. The method of claim 26, wherein the time period is 30 minutes or less.

28. The method of claim 26, wherein the time period is 10 minutes or less.

29. The method of claim 26, wherein the time period is 5 minutes or less.

30. The method of claim 26, wherein the time period is 1 minute or less.
